(19) United States
(12) Patent Application Publication
(10) Pub. No.: US 2013/0289438 A1
(43) Pub. Date: Oct. 31, 2013

(54) Weight Loss Method and Apparatus

(71) Applicant: Angela Marie Lyon, Sandy, UT (US)

(72) Inventor: Angela Marie Lyon, Sandy, UT (US)

(73) Assignee: CORE FITNESS AND WELLNESS, LLC, Sandy, UT (US)

(21) Appl. No.: 13/459,171

(22) Filed: Apr. 28, 2012

(51) Int. Cl. A61M 37/00; F25D 3/08; A45F 3/00; A61F 7/10; A61B 5/01

(52) U.S. Cl. 600/549; 607/112; 604/304; 224/660; 62/457.2

(57) ABSTRACT

Home-use apparatuses for removing heat from body-fat rich tissues and related devices, systems, and methods. One embodiment of the disclosed technology includes a belt which can be worn around a part of the user's body were the user desires to lose body fat. The belt has a pocket which holds a cold pack. The cold pack cools the user's skin and reduces body-fat rich cells in the area where cooling in applied. Because the belt is made of light, flexible material and has no burdensome mechanical or electric parts, the user retains flexibility and mobility while wearing the belt.

No federal grant money was used to support the research that contributed to the invention.

### TECHNICAL FIELD

The present application relates to home-use apparatuses for removing heat from body-fat rich tissues and related devices, systems, and methods. In particular, embodiments of the present invention include apparatuses that are simple, portable, easy to use, and that may be recharged or regenerated by a conventional freezer.

### BACKGROUND OF THE INVENTION

Body fat can accumulate in many parts of the human body, especially in subcutaneous tissue directly beneath the skin. This tissue, sometimes called adipose tissue, forms a layer of lipid-rich cells which contain a higher concentration of lipids compared to other, non-lipid-rich cells. Accumulation of too many of these lipid-rich cells in the human body is generally undesired because it contributes to an outward appearance that is generally thought to be unattractive, it contributes to the appearance of cellulite on the skin, and it is associated with health risks.

A variety of invasive and noninvasive procedures have been used for removing excess body fat. Invasive procedures include surgeries such as liposuction, laser-assisted liposuction, and mesotherapy. Some disadvantages to these invasive procedures are expense, need for hospitalization, need for anesthesia, and a period of recovery after the procedure. Some traditional noninvasive procedures for removing excess body fat include topical agents, weight-loss drugs, exercise, and dieting. These methods may take a long time before producing a noticeable effect on body fat. Exercise and dieting require willpower and exertion, which can make it difficult for individuals to continue them long enough to remove the desired amount of body fat. Individuals who are injured or sick may not be able to exercise. Also, topical agents and weight-loss drugs can produce allergic reactions in some people or have other undesirable side effects. Furthermore, it may not be possible to target fat loss in a certain area of the body using systemic weight loss methods.

Newer methods for reducing body fat include methods that apply electromagnetic radiation to the tissue, as in U.S. Patent Publication No. 2006/0036300 and U.S. Pat. No. 5,143,063, and methods that use high intensity focused ultrasound as in U.S. Pat. Nos. 7,258,674 and 7,347,855. Methods have now been developed that use cooling to reduce body fat, such as in U.S. Pat. No. 7,367,341, U.S. Pat. Pub. No. 2005/0251120, and U.S. Pat. Pub. No. 2011/0238051. These methods take advantage of a property of lipid-rich tissue that allows it to be destroyed by cooling at temperatures which do not damage the surrounding non-lipid-rich tissue. Some of these cooling methods require expensive and complicated electronic and/or mechanical systems.

### BRIEF SUMMARY OF THE DRAWINGS

FIG. 1A is an illustration of a human having applied lotion to a part of the human body (in this case, the abdomen) in accordance with an embodiment of the disclosure.

FIG. 1B is an illustration of a human having applied the lotion and a plastic wrap to a part of the human body (in this case, the abdomen) in accordance with an embodiment of the disclosure.

FIG. 1C is an illustration of a human having applied the lotion, the plastic wrap, and a cold pack to a part of the human body (in this case, the abdomen) in accordance with an embodiment of the disclosure.

FIG. 1D is an illustration of a human having applied the lotion, the plastic wrap, the cold pack, and a belt to a part of the human body (in this case, the abdomen) in accordance with an embodiment of the disclosure.

FIG. 1E is an illustration of a frontal view of the human from FIG. 1D, having applied the lotion, the plastic wrap, the cold pack, and the belt to a part of the human body (in this case, the abdomen) in accordance with an embodiment of the disclosure.

FIG. 2A is an illustration of a side view of a belt with a pocket containing a cold pack in accordance with an embodiment of the disclosure.

FIG. 2B is an illustration of an interior-surface-facing view of the belt of FIG. 2A containing the cold pack in accordance with an embodiment of the disclosure.

FIG. 2C is an illustration of an exterior-surface-facing view of the belt of FIG. 2A containing the cold pack in accordance with an embodiment of the disclosure.

FIG. 2D is an illustration of a cross sectional top-down view of a human abdomen with lotion that has been applied to a pocket of a belt, and the belt circumscribes the human abdomen and a cold pack is disposed inside the pocket.

FIG. 3A is an illustration of a step in a method in accordance with an embodiment of the disclosure, the step comprising positioning a belt behind a human back.

FIG. 3B is an illustration of a step in a method in accordance with an embodiment of the disclosure, the step comprising wrapping a first end of the belt around a human abdomen.

FIG. 3C is an illustration of a step in a method in accordance with an embodiment of the disclosure, the step comprising wrapping the second end of a belt around the human abdomen.

FIG. 4A is an illustration of a step in a method in accordance with an embodiment of the disclosure with a top-loading pocket of a belt, the step comprising removing a cold pack from the top-loading pocket.

FIG. 4B is an illustration of a step in a method in accordance with an embodiment of the disclosure, the step comprising positioning the cold pack of FIG. 4A between the pocket and the belt.

FIG. 4C is an illustration of a step in a method disclosed in FIG. 4B in accordance with an embodiment of the disclosure.

### DETAILED DESCRIPTION OF THE INVENTION

### 1. Overview

Numeric ranges recited herein are inclusive of the numbers defining the range and include and are supportive of each integer within the defined range. Unless otherwise noted, the terms “a” or “an” are to be construed as meaning “at least one of.” The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. Unless otherwise specified, the steps of a method may be conducted in any order.

Several specific embodiments of methods and apparatuses in accordance with the disclosed technology are described below. The described embodiments include many details to enable a person of skill in the art to practice the embodiments. However, many of the details of the disclosed specific embodiments may not be necessary to practice other embodiments of the disclosed technology. The disclosed technology may include methods and apparatuses that are covered by the claims but not specifically disclosed in the specification.

References throughout this specification to “one embodiment,” “an embodiment,” “at least one embodiment,” and the like mean that the disclosed features may be contained in a particular embodiment of the disclosed technology. All such references may refer to separate, specific embodiments, and these references do not necessarily all refer to the same embodiment. Any disclosed steps, features, structures, characteristics, and details may be combined in any suitable way, and the disclosed technology includes all such combinations that are covered by the claims.

One embodiment of the disclosed technology includes an apparatus for holding a cold source in heat transfer contact with the human skin. The apparatus may include a belt which straps the apparatus to the part of the human body where fat loss is desired. The cold source may be a passive cooling device, such as a cold pack.

Another embodiment includes a method for reducing body fat by applying cold to a part of the human body. This method may include the steps of applying a passive cooling device, such as a cold pack, to the skin, holding the cold pack in place by strapping it on with a belt, and waiting for a period of time before removing the belt and cold pack. Other, more specific descriptions of apparatuses and methods that are also embodiments of the disclosed technology are given below in this specification.

Methods for reducing body fat by using cold are hypothesized to work because of the phenomenon that at certain low temperatures, lipid-rich cells are selectively reduced in comparison to other, non-lipid-rich cells. The exact mechanism through which lipid-rich cells are selectively reduced is unknown, but it is believed that the cold causes the lipid-rich cells to shrink, die, or be removed by the body. This is possibly accomplished by altering the cells in a way that triggers an apoptotic cascade. Apoptosis is also known as “programmed cell death” and is natural mechanism by which the body causes cells to self-destruct. Apoptotic cell death is different from necrotic cell death (another mechanism by which cells in the body can die) in that apoptotic cell death does not cause inflammation in the surrounding tissue, but necrotic cell death does cause inflammation. Therefore, removing lipid-rich cells by inducing apoptosis may reduce body fat without causing symptoms of inflammation, such as pain and swelling.

Selective cold-induced apoptosis of lipid-rich cells might occur through one or a combination of several suspected mechanisms. One possible mechanism involves crystallization of the lipids within the adipocytes, which damages the cells internally and triggers apoptosis. This crystallization occurs at temperatures which do not cause crystallization of non-lipid-rich cells. According to another possible mechanism, cooling causes a phase transition in the cell's bi-lipid membrane, and this disrupts the membrane and triggers apoptosis. In another possible mechanism, cooling causes the cell membrane to stop transporting ions correctly, and this causes an ion imbalance across the cell membrane, leading to apoptosis. Also, local cold exposure is believed to cause increased metabolism of fat in the exposed area. Any of these mechanisms or a combination of them could encourage body fat reduction. Each of the specific embodiments disclosed below uses locally applied cooling to cause fat loss by one or more of these mechanisms, or by other, unknown mechanisms.

### 2. Representative Embodiments

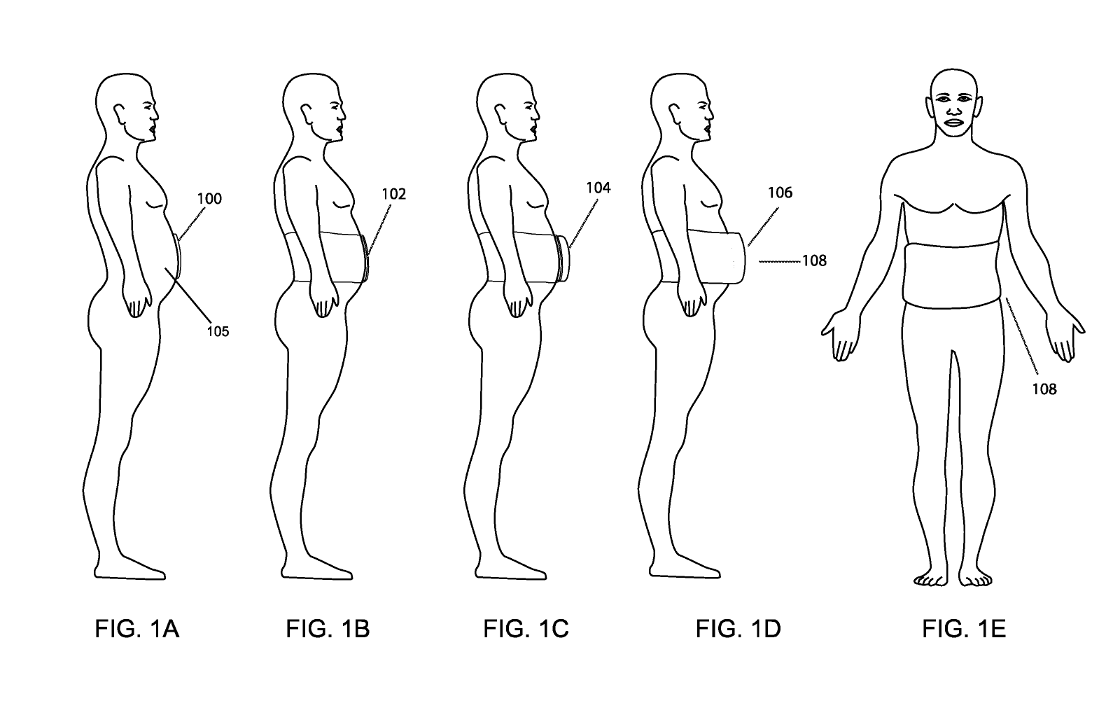

FIG. 1A through FIG. 1E show steps in a method for reducing body fat by applying local cooling in accordance with an embodiment of the disclosed technology. FIG. 1A shows the step of applying a layer of lotion 100 to an area of the human body where body fat loss is desired (in this case a body part such as an abdomen 105). FIG. 1B shows the step of wrapping one end of plastic wrap 102 around the abdomen 105 so that it overlaps the plastic wrap 102. FIG. 1C shows the step of applying a cold pack 104 to the abdomen 105 so that the distance between the cold pack 104 and the abdomen 105 is sufficiently short so that the cold pack 104 can accept the transfer of heat from the abdomen 105. FIG. 1D shows the step of wrapping a belt 106 around the cold pack 104, the plastic wrap 102, the lotion 100, and the abdomen 105 to hold the cold pack 104 in place, that is, keep the cold pack 104 in the relatively same location. The belt 106, the cold pack 104, the plastic wrap 102, and the lotion 100 together form an apparatus 108 for reducing body fat by local cooling. FIG. 1E shows a frontal view of a user of the apparatus 108 after the user has performed the step of wrapping a belt 106 around the cold pack 104, plastic wrap 102, lotion 100, and abdomen 105, and forming a complete apparatus 108 for reducing body fat by local cooling.

In at least one embodiment the apparatus 108 comprises plastic wrap 102; examples of plastic wrap 102 include thin plastic film such as Saran Wrap® plastic wrap. In at least one embodiment, the user can perform, in order, the steps of applying lotion 100 to the desired body part, wrapping plastic wrap 102 around the body part, applying a cold pack 104 to the body part, and wrapping a belt 106 around the cold pack 104, plastic wrap 102, lotion 100, and body part. In another embodiment, the user can first apply lotion 100 to the plastic wrap 102, and then apply the plastic wrap 102 with lotion 100 to the desired body part. In yet another embodiment, the plastic wrap 102 may come with lotion pre-applied so the user can apply the plastic wrap 102 with lotion without needing to perform the step of applying lotion 100. In some embodiments any thin material such as aluminum foil is substituted for the plastic wrap 102.

In at least one embodiment the user performs the steps of applying all four components: lotion 100, plastic wrap 102, cold pack 104, and belt 106. But in at least one other embodiment the user only performs the steps of wrapping the body part with the plastic wrap 102, applying the cold pack 104, and wrapping with the belt 106 but does not perform the step of applying lotion 100. In yet another embodiment the user performs only the steps of applying a cold pack 104 and wrapping a belt 106 so that it surrounds the cold pack 104 and overlaps itself, but not the step of applying lotion 100 or the step of wrapping the lotion and body part with plastic wrap 102. In another embodiment the user can apply a layer of lotion 100 to a body part 105 such as an abdomen, apply a cold pack 104 to the layer of lotion, and wrap the cold pack and body part 105, such as an abdomen, with a belt 106, without wrapping the abdomen with plastic wrap 102.

In at least one embodiment, the user first performs the step of wrapping a belt 106 around the desired body part, and then performs, in any order, the steps of applying lotion 100 to the body part, sliding plastic wrap 102 between the body part, such as an abdomen 105, and the belt 106, and sliding a cold pack 104 between the body part and the belt 106, so that the plastic wrap 102 is between the cold pack 104 and body part. In another embodiment, the user first applies lotion 100 to the body part, then wraps a belt 106 around the body part, then slides plastic wrap 102 and a cold pack 104 between the body part and the belt 106, such that the plastic wrap 102 is between the body part and the cold pack 104.

Without detracting from the possibilities of order and selection of steps to be performed in specific embodiments as explained above, using all four components (lotion 100, plastic wrap 102, cold pack 104, and belt 106) may provide several advantages. Lotion may enhance the heat transfer between the user's skin and the cold pack, both because lotion may have a high heat transfer coefficient and because it can eliminate air bubbles between the plastic wrap and skin and provide a better seal and better contact for heat transfer. The lotion may also contain additives to provide additional effects. This might include additives configured to decrease the surface temperature of human epidermis cells, additives configured to increase metabolic activity in humans, additives configured to decreasing the sensitivity of human epidermis to temperature changes, additives configured to suppress appetite, and combinations thereof. For example, nonlimiting examples of additives may include oils derived from plants, oils derived from fruits, oils derived from citrus fruits, peppermint oil, spearmint oil, eucalyptus, clove oil, oil of wintergreen, cinnamon, cinnamon aldehyde, menthol, oxygenated p-menthane derivatives, p-menthane alcohols, camphor, monomenthyl succinate, monomenthyl glutarate, menthol ethylene glycol carbonate, menthone glycerin acetal, WS-1, esters of WS-1, N-substituted amides of WS-1, WS-3, WS-14, menthyl succinate, menthyl lactate, 3-1-menthoxypropane-1,2-diol, menthone glycerol ketals, N-substituted p-methane carboxamide, menthol, peppermint essential oil, menthol essential oil, grapefruit essential oil, ethanol, and combinations thereof. Using plastic wrap may also provide the advantage of protecting the skin from direct contact with the cold pack, which could damage the skin cells where the cold pack is applied if the cold pack is too cold. The plastic wrap may slow down heat transfer sufficiently to allow the skin to be cooled safely. Plastic wrap also provides the advantage of protecting the cold pack and belt from lotion that has been applied to the skin, so that the cold pack and belt remain clean and do not need to be washed between uses.

The lotion, plastic wrap, cold pack, and belt used in the embodiments described above may have any of the features, structures, characteristics, or details disclosed in other embodiments in this specification, including apparatuses disclosed in embodiments below.

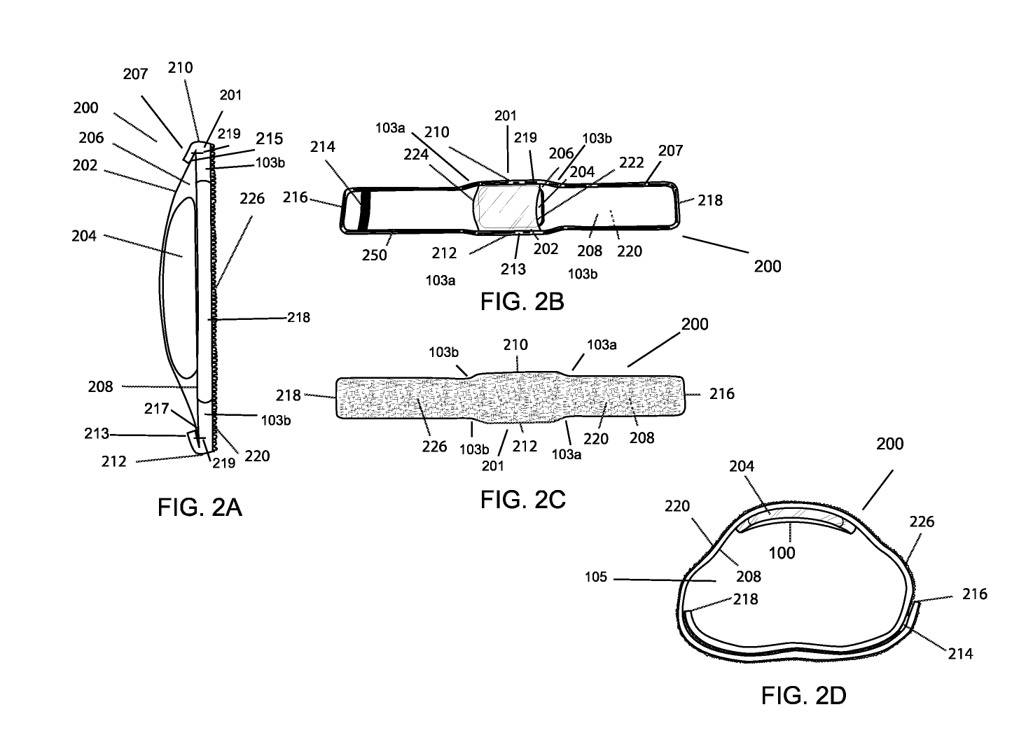

FIG. 2A shows a side view of an apparatus in accordance with an embodiment of the disclosed technology. For directional reference, from the viewpoint of FIG. 2A the longitudinal axis extends out of the page and back into the page, the vertical axis extends in the up and down directions, and the lateral axis extends in the left and right directions. A belt 200 includes a pocket 202 defining an interior space 206. The upper edge 210 of the belt comprises an upper hem 207 that is affixed to an upper edge of a pocket (pocket upper edge 215) which is affixed to the interior surface 208 of the pocket 202. The upper hem 207 of the upper edge 210 folds over the pocket upper edge 215; the pocket upper edge 215 is affixed to the interior surface 208. The lower hem 213 of the lower edge 212 folds over the pocket lower edge 217; the pocket lower edge 217 is affixed to the interior surface 208. The belt 200 has an upper edge 210, a lower edge 212, an interior surface 208, and an exterior surface 220. The upper edge 210 comprises the upper edge of the interior surface and the upper edge of the exterior surface. The lower edge 212 of the belt 200 also comprise the lower edge of the interior surface 208 and lower edge of the exterior surface 220. The upper edge 210 and the lower edge 212 extend along the longitudinal axis. A cold pack 104 is disposed inside the interior space 206 of the pocket 202. The pocket 202 is fixedly attached to the belt 200 along the upper edge 210 and lower edge 212 of the interior surface 208. The exterior surface 220 is covered by a loop fastener 226 of a hook and loop fastener. In some embodiments the pocket is made from a fabric material. In some embodiments the pocket is completely detachable from the belt.

FIG. 2B shows an interior-surface-facing view of the belt 200. For directional reference, from the viewpoint of FIG. 2B the longitudinal axis extends in the left and right directions, the vertical axis extends in the up and down directions, and the lateral axis extends out of the page and back into the page. The belt 200 has a first end 216 and a second end 218. In some embodiments the edges and ends of the belt 200 define the perimeter of the belt 200, including an upper edge of the belt 200 (belt upper edge 210), a lower edge 212 of the belt 200 (belt lower edge 212), and the first end 216 and second end 218, which are all finished with a hem 250. In this particular embodiment, the hem 250 is not exclusively composed of fabric of the belt 200. The hem 250 ranges between 0.1 cm and 15 cm in height and in some embodiments is around 1 cm in height; the hem 250 is taken from the edges that form the perimeter of the belt 200 and folded over the pocket 202. In an embodiment the pocket 202 is affixed to the interior surface 208 of the belt 200, then affixed to the interior surface 208 of the belt 200. In some embodiments the hem 250 runs along the portions of the belt that are in contact with the pocket 202. In some embodiments an industrial sewing machine is used to stitch the pocket upper edge 215 to the interior surface 208 of the belt 200 and to the hem 250. Rayon thread, polyester thread, or the thread of some other material can be used to form stitches 219 which affix the pocket upper edge 215 to the interior surface 208 of the belt 200 and to the hem 250; however, other materials such as glue or staples can be used to affix the pocket 215 to the interior surface 208 of the belt 200 and to the hem 250. The pocket 202 is disposed at or near the middle of the belt 200, between the first end 216 and second end 218. In this particular embodiment, the pocket 202 is a rectangular sheet of transparent plastic that has a length of 31.5 cm and a height of 18.2 cm at the ends and 20 cm at the middle section. In some embodiments the thickness of the plastic is 0.001 mm; in other embodiments the thickness is 10 mm; in other embodiments the thickness is 0.2 mm; any thickness of plastic can be used so long as heat can be transferred from a cold pack that is situated behind the plastic to a body part that is contacting the other side of the plastic. In this particular embodiment the pocket 202 is a plastic sheet that is attached to the belt upper edge 210 and belt lower edge 212 by placing the plastic sheet between the upper 210 and lower 212 edges of the belt 200 and sewing seams through the belt upper edge 210, the plastic sheet, and the belt lower edge 212. The pocket 202 has a first edge 224 (pocket first edge 224) and a second edge 222 (pocket second edge 222). In this embodiment the pocket first edge 224 and the pocket second edge 222 of the pocket 202 are not attached to the belt 200, thereby forming openings at the first edge 224 and second edge 222 which open into the interior space 206 inside the pocket 202. The cold pack 104 is disposed inside the interior space 206, in some embodiments at or near the middle of the pocket 202 and between the openings at the first edge 224 and second edge 222. A hook fastener 214, such as Velcro® hoops, of a hook and loop fastener is disposed near the first end 216 of the belt 200, on the interior surface 208 of the belt 200. In some embodiments the belt 200 is made of neoprene fabric, which imparts to the belt 200 stretchability. In some embodiments a middle region 201 of the belt 200 is the area of the belt 200 facing the pocket 202; in some embodiments the middle region 201 of the belt has a height which is greater than the height of the first end 216 and the height of the second end 218 of the belt 200. In some embodiments tapered sections 103a, 103b of the belt 200 taper to a smaller height than the height of the middle region 201 so that at least one of the heights selected from the group consisting of the height of the first end 216 and the height of the second end 218 is less than the height of the middle region 201. Tapered section 103a of the belt may be located between the first end 216 and the middle region 201; tapered section 103b may be located between the second end 218 and the middle region 201. The tapered sections 103a, 103b allow the first end 216 and the second end 218 to have smaller heights than the middle region 201 the belt 200. In some embodiments tapered sections 103a, 103b are replaced with a vertical sections of the belt upper edge 210 and the belt lower edge 212 such that the middle region 201 is connected by a vertical, non-tapering section of the belt upper edge 210 to the first end 216, by a vertical, non-tapering section of the belt upper edge 210 to the second end 218, by a vertical, non-tapering section of the belt lower edge 212 to the first end 216, and by a vertical, non-tapering section of the belt lower edge 212 to the second end 218. In other embodiments the middle region 201 has a greater width than the pocket 202; in other embodiments the middle region 201 has a smaller width than the pocket 202. The narrower dimension, that is the smaller heights, of the first end 216 and the second end 218 allows the first end 216 and the second end 218 to have greater stretchability. In other embodiments the middle region 201, the first end 216, and the second end 218 are the same height and the belt does not have tapering sections 103a, 103b.

FIG. 2C shows an exterior-surface-facing view of the belt 200. For directional reference, from the viewpoint of FIG. 2C the longitudinal axis extends in the left and right directions, the vertical axis extends in the up and down directions, and the lateral axis extends out of the page and back into the page. A loop fastener is comprised of more than one loop. In a preferred embodiment a loop fastener 226, such as Velcro® loops, covers the exterior surface 220, allowing for the hook fastener 214 to attach to the loop fastener 226 at any location along the length of the belt 200, allowing the belt 200 to be wearable by various users with various waist sizes. (The belt can be configured for different body parts of men, women, adolescents, and children. When determining the dimensions of the belt, one may consult demographic data to determine the average size of specific body parts for different segments of the population, such as the average circumference of a thigh belonging to a female who is between 18 and 25 years of age. The belt may then be designed to be of sufficient length so that it can wrap at least one and a half times around the thigh of an obese female who is between the ages of 18 and 25 years of age.) In this particular embodiment, a hook fastener 214 has a length of between 1 to 2 inches and is located on the interior surface 208 at the first end 216. However, in other embodiments the loop fastener 226 does not extend along the entire exterior surface 220 of the belt but forms a particular region of the belt 200. In some embodiments the interior surface has a loop fastener and the exterior surface comprises a hook fastener. In other embodiments the loop fastener and hook fastener are replaced with any type of fastener such as buttons or zippers and may be detachable.

FIG. 2D shows a top-down, cross sectional view of the belt 200 which is wrapped around a human abdomen. The hook fastener 214 is attached to the loop fastener 226, securing the belt 200 around the human abdomen. The belt 200 holds the cold pack 104 in place against the front of the abdomen, or belly area. A layer of lotion 100 is applied to the pocket 202. In the embodiment depicted in FIG. 2D the second end 218 is first wrapped around the body part so that the lotion 100 of cold pack 104 is positioned on the human abdomen and the cold pack 104 is within close proximity of the abdomen so that heat may be transferred from the abdomen to the cold pack 104. Although the abdomen is shown as an illustrative example of a body part 105, many other body parts could be used with the methods and apparatus that are disclosed herein. For example, the apparatus could be wrapped around a thigh or a forearm or even the neck.

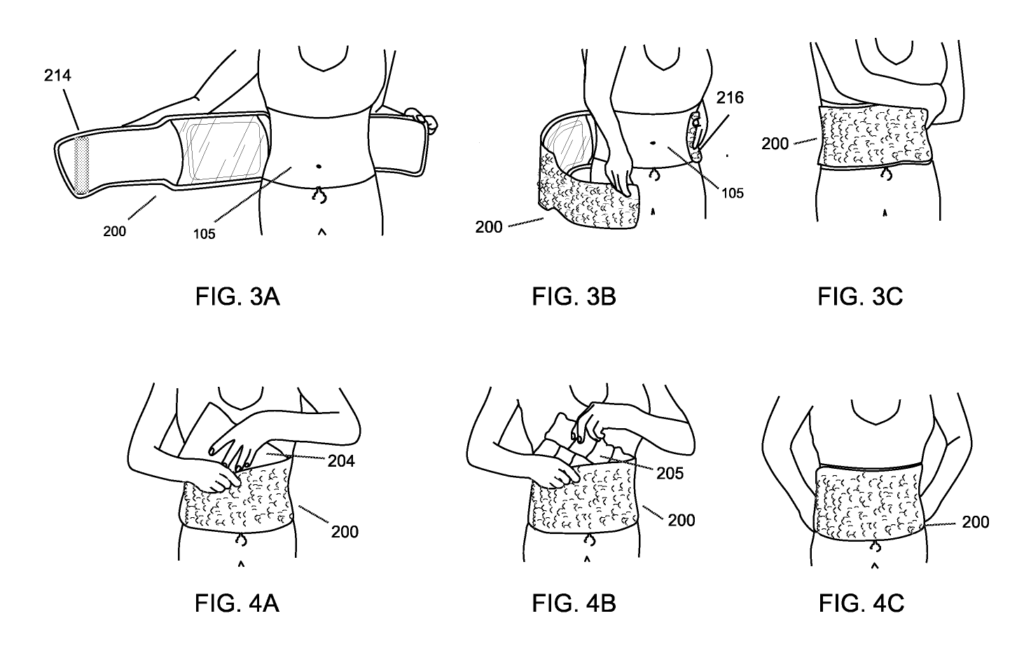

FIG. 3A through FIG. 3C show steps in a method for reducing body fat by applying local cooling in accordance with an embodiment of the disclosed technology. FIG. 3A shows the step of holding a belt 200, such as the apparatuses disclosed in FIG. 2, in a position ready to be wrapped around a human body part 105 (in this case, the abdomen). FIG. 3B shows the step of wrapping the belt 200 around the body part 105. FIG. 3C shows the step of fastening the belt 200. In this embodiment, the belt 200 may be fastened by affixing a hook fastener 214 to a loop fastener 226 and securing the belt 200 around the abdomen 105. The belt 200 may be made from neoprene, which imparts to the belt stretchability. This allows the user to stretch the belt 200 slightly before fastening the belt 200. The slightly stretched belt 200 will then stay in place securely on the body part 105. The security and flexibility of the belt 200 allow the user to move the body part 105 while wearing the belt 200 not having to fear that the belt 200 will move out of its place or restrict the movement of the user. In some embodiments the surface skin temperature is reduced to 40 degrees. In other embodiments, the surface skin temperature is reduced to less than 61 degrees but greater than 40 degrees.

FIG. 4A through FIG. 4C show steps in a method for reducing body fat by applying local cooling in accordance with an embodiment of the disclosed technology. In this particular embodiment, the belt 200 has a pocket with an opening at the top edge, so that the user can slide a cold pack 104 into the pocket from the top while wearing the belt. FIG. 4A shows the step of removing the cold pack 104 from the pocket. FIG. 4B shows the step of rotating the cold pack 104 approximately 180 degrees around a vertical axis so that the side of the cold pack 104 that was contacting the interior surface of the belt 200 is contacting the body part. FIG. 4C shows the step of waiting for a period of time while wearing the belt 400 with the cold pack 404 inserted. In some embodiments, users wear the belt 400 with the cold pack 404 inserted for one minute before taking off the cold pack; in other embodiments, users wear the belt 400 with the cold pack 404 inserted for 10 minutes before taking off the belt 400 with the cold pack 404 inserted; in other embodiments the user wears the cold pack for up to 50 minutes before taking off the belt 400 with the cold pack 404 inserted; in other embodiments, the user wears the belt 400 with the cold pack 404 inserted for at least 50 minutes at least five different times during a day; in some embodiments the users wear the belt 400 with the cold pack 404 inserted only once a week; in other embodiments, the user wears the belt 400 with the cold pack 404 inserted every day.

In one embodiment, the user fastens the belt 400, then waits for at least three minutes but no more than fifty minutes, then removes the cold pack 404 from the pocket in the belt 400, flips the cold pack over, reinserts it into the pocket, and then waits an additional at least three minutes but no more than fifty minutes. In another embodiment, the user wears the belt with a first cold pack 404 for a period of time, then removes the first cold pack 404 from the pocket 202 and inserts a second cold pack in its place. In yet another embodiment, the user places multiple, but not more than three, cold packs into the pocket 202. The user can flip the multiple cold packs over after a period of time, or remove the multiple cold packs all together and replace them with other multiple cold packs. Or the user can replace the cold packs at staggered intervals, removing one or two at a time and replacing them with one or two other cold packs. By using combinations of the waiting periods, numbers of cold packs, flipping of cold packs, and replacing of cold packs as described in the embodiments above, the user can achieve a particular temperature at the surface of the skin of the body party on which the belt is worn, and maintain the temperature nearly constant for any period of time. Or, by using a single cold pack without replacing or flipping it, the user can cause the surface of the skin to slowly increase in temperature as the cold pack 404 increases in temperature over time.

In this particular embodiment the cold pack 404 is a segmented cold pack, such as Techni Ice. Various other types of cold packs 404 may be used in other embodiments. For example, the cold pack 404 could be a portion of dry ice, a dry ice pack, a gel pack, ice, blue ice, a dehydrated refrigerant sheet, a plastic bag comprising ice substitute, and combinations thereof. Various types of cold packs 404 may provide various benefits, such as having different phase transition temperatures, temperature profiles, flexibility, heat capacity, and reusability. A segmented cold pack such as Techni Ice provides the advantage that the segments may be cut apart in such a way as the shape the ice pack for a particular body part. For example, a user could cut the segmented ice pack into an “L” shape when applying it to a knee or elbow joint. In some embodiments the user applies lotion and then inserts a cold gel pack into the pocket of the apparatus, contacts the pocket of the apparatus to a body part such as an abdomen, and secures the belt. A gel pack is a pliable bag containing a gel material that can be cooled or frozen; gel packs are reusable and pliable. See for example the 3M® Hot Cold Pack or the Caldera® Therapy Gel Pack. Other examples of cold packs include bags comprising plastic and a cap that can be filled with ice. Another example of a cold pack is a bag of frozen vegetables, such as peas. Even another example of a cold pack is an amount of ice or an amount of ice contained within a plastic sealable bag.

In one embodiment, the user inserts a thermometer between the belt and the skin to measure the temperature of the skin. In one embodiment, the temperature of the skin is between 40 and 60 degrees Fahrenheit. In another embodiment, the temperature of the skin is below 40 degrees Fahrenheit.

The disclosed technology can be used to reduce body fat by applying local cooling to any part of the body where fat loss is desired. Thus, embodiments of the disclosed technology include belts configured to be worn on any part of the human body. For example, a belt could be worn to provide local cooling to the abdomen, back, neck, waist, torso, trunk, hip, buttock, chest, arm, forearm, thigh, knee, face as measured from the chin to the first cheek to the portion of the scalp above the forehead to the second cheek and to the chin, legs, and calf. In some embodiments the belt has a length greater than 4% but less than 170% of the circumference of the body part on which it is intended to be worn. In one particular embodiment, the belt is 8 inches high and 40 inches long. Belts in accordance with the disclosed technology may have other lengths and heights, depending on the body part on which it is intended to be worn and the size and age of the user. Belts may be configured to fit any body part of any user, male or female, of any age, at any height and weight.

A preliminary test study was completed with five to six subjects who used the apparatus on their abdomen with a Techni-Ice cold pack for approximately 30 min before rotating the Techni-Ice cold pack so that the side which was facing away from the abdomen was then facing the abdomen. The average decrease in waistline size of subject's who used the belt was 2 inches; all subjects also lost some weight. In the test study, the waistline of each subject decreased so that it was noticeable that the subject had lost at least 2 inches. At least one of the subjects noticed the decrease in the size of the subject's waistline after 5 days; at least one of the subjects noticed the decrease in the size of the subject's waistline after 2 weeks. One subject lost 7 pounds and the size of the subject's waistlines decreased approximately 2 inches after using the belt for 6 weeks. Another subject lost 2 inches of waistline and 4 pounds of body weight after using the belt on two separate occasions in the same area with a 14 day interval between the occasions. This was the results after using the product only once every 14 days in same area. During the preliminary study, subjects contacted one of three body areas with the pocket of the belt: the abdomen, the left side of the waist known in the vernacular as a “love handle”, and the right side of the waist known in the vernacular as a “love handle”. The pocket of the belt was cold because the pocket of the belt was in direct contact with a cold pack that had a surface temperature that was less than skin surface temperature.

It should be appreciated that the apparatuses and methods of the present invention are capable of being incorporated in the form of a variety of embodiments, only a few of which have been illustrated and described above. The invention may be embodied in other forms without departing from its spirit or essential characteristics. The described embodiments are to be considered in all respects only as illustrative and not restrictive, and the scope of the invention, therefore, indicated by the appended claims rather than by the foregoing description. All changes which come within the meaning and range of equivalency of the claims are to be embraced within their scope.

What is claimed is:

1. An apparatus comprising:
a belt comprising
an interior surface;
an upper long edge of said interior surface;
a lower long edge of said interior surface;
a first short edge of said interior surface; and,
a second short edge of said interior surface;
an exterior surface;
a lateral axis;
a vertical axis; and,
a longitudinal axis;
a pocket defining an inner space, extending along a portion of the interior surface along the longitudinal axis of said belt, said pocket comprising:
a first edge;
a second edge;
an upper edge; and,
a lower edge wherein at least one affixed edge is affixed to said belt and selected from the group consisting of said first edge, said second edge, said upper edge, and said lower edge; and,
at least one fastener.

2. The apparatus of claim 1 wherein said at least one affixed edge consists of two affixed edges selected from the group consisting of said first edge, said second edge, said upper edge, and said lower edge, wherein said two affixed edges are affixed to said belt.

3. The apparatus of claim 1 wherein said at least one affixed edge consists of three affixed edges selected from the group consisting of said first edge, said second edge, said upper edge and said lower edge, wherein said three affixed edges are affixed to said belt.

4. The apparatus of claim 2 wherein said two fixed edges consists of said upper edge and said lower edge.

5. The apparatus of claim 4 wherein the upper long edge of said exterior surface comprises an upper hem and the lower long edge of said exterior surface comprises a lower hem wherein said upper hem is affixed to said upper edge of said pocket and said upper edge of said pocket is affixed to said upper long edge of said interior surface and said lower hem is affixed to said lower edge of said pocket and said lower edge of said pocket is affixed to said lower long edge of said interior surface.

6. The apparatus of claim 3 wherein said three affixed edges consists of said first edge, said lower edge, and said second edge.

7. The apparatus of claim 2, wherein at least one affixed edge is detachable.

8. The apparatus of claim 4 further comprising at least one cold pack disposed inside the pocket.

9. The apparatus of claim 8 wherein the at least one cold pack is selected from the group consisting of a portion of dry ice, a dry ice pack, a gel pack, ice, a dehydrated refrigerant sheet, a plastic bag comprising ice substitute, and combinations thereof.

10. The apparatus of claim 9 wherein the at least one cold pack comprises no more than three cold packs.

11. The apparatus of claim 10 wherein said pocket comprises a thin panel comprising material selected from the group consisting of plastic, fabric, and combinations thereof.

12. The apparatus of claim 11 wherein the length of said belt is selected from the group consisting of lengths greater than 4% but less than 170% the circumference of a body part selected from the group consisting of a waist of a human, a torso of a human, a trunk of a human, a hip of a human, a buttock of a human, a chest of a human, an arm of a human, a forearm of a human, a thigh of a human, a knee of a human, a face of a human as measured from the chin to the first cheek to the portion of the scalp above the forehead to the second cheek and to the chin, legs, and a calf of a human.

13. The apparatus of claim 11 wherein the height of said belt is substantially 8 inches and the length of said belt is substantially 40 inches in length.

14. The apparatus of claim 12 wherein the length of said pocket is greater than a ½ inch and less than 40 inches, the height of said pocket is greater than a ½ inch and less than 24 inches, and the width of said pocket is greater than 0.025 inches and less than ¼ inches.

15. The apparatus of claim 14 further comprising a layer of lotion affixed to the exterior surface of said thin plastic panel.

16. The apparatus of claim 15 wherein said layer of lotion comprises at least one additive selected from the group consisting of additives configured to decrease the surface temperature of human epidermis cells, additives configured to increase metabolic activity in humans, additives configured to decreasing the sensitivity of human epidermis to temperature changes, additives configured to suppress appetite, and combinations thereof.

17. The apparatus of claim 16 wherein the at least one additive is selected from the group consisting of oils derived from plants, oils derived from fruits, oils derived from citrus fruits, peppermint oil, spearmint oil, eucalyptus, clove oil, oil of wintergreen, cinnamon, cinnamon aldehyde, menthol, oxygenated p-menthane derivatives, p-menthane alcohols, camphor, monomenthyl succinate, monomenthyl glutarate, menthol ethylene glycol carbonate, menthone glycerin acetal, WS-1, esters of WS-1, N-substituted amides of WS-1, WS-3, WS-14, menthyl succinate, menthyl lactate, 3-1-menthoxypropane-1,2-diol, menthone glycerol ketals, N-substituted p-methane carboxamide, menthol, peppermint essential oil, menthol essential oil, grapefruit essential oil, ethanol, and combinations thereof.

18. The apparatus of claim 15 wherein said human is selected from the group consisting of an eight-year old male who is at the 50^{th} percentile in height but at the 60^{th} percentile in weight, an eight-year old male who is at the 50^{th} percentile in height but at the 70^{th} percentile in weight, an eight-year old male who is at the 50^{th} percentile in height but at the 80^{th} percentile in weight, an eight-year old male who is at the 50^{th} percentile in height but at the 90^{th} percentile in weight, an eight-year old male who is at the 50^{th} percentile in height but at the 99^{th} percentile in weight, an adolescent male who is at the 50^{th} percentile in height but at the 60^{th} percentile in weight, an adolescent male who is at the 50^{th} percentile in height but at the 70^{th} percentile in weight, an adolescent male who is at the 50^{th} percentile in height but at the 80^{th} percentile in weight, an adolescent male who is at the 50^{th} percentile in height but at the 90^{th} percentile in weight, an adolescent male who is at the 50^{th} percentile in height but at the 99^{th} percentile in weight, an adult male who is at the 50^{th} percentile in height but at the 60^{th} percentile in weight, an adult male who is at the 50^{th} percentile in height but at the 70^{th} percentile in weight,
an adult male who is at the 50^{th} percentile in height but at the 80^{th} percentile in weight, an adult male who is at the 50^{th} percentile in height but at the 90^{th} percentile in weight, an adult male who is at the 50^{th} percentile in height but at the 99^{th} percentile in weight, a middle-aged male who is at the 50^{th} percentile in height but at the 60^{th} percentile in weight, a middle-aged male who is at the 50^{th} percentile in height but at the 70^{th} percentile in weight, a middle-aged male who is at the 50^{th} percentile in height but at the 80^{th} percentile in weight, a middle-aged male who is at the 50^{th} percentile in height but at the 90^{th} percentile in weight, a middle-aged male who is at the 50^{th} percentile in height but at the 99^{th} percentile in weight, an elderly male who is at the 50^{th} percentile in height but at the 60^{th} percentile in weight, an elderly male who is at the 50^{th} percentile in height but at the 70^{th} percentile in weight, an elderly male who is at the 50^{th} percentile in height but at the 80^{th} percentile in weight, an elderly male who is at the 50^{th} percentile in height but at the 90^{th} percentile in weight, an elderly male who is at the 50^{th} percentile in height but at the 99^{th} percentile in weight, an eight-year old female who is at the 50^{th} percentile in height but at the 60^{th} percentile in weight, an eight-year old female who is at the 50^{th} percentile in height but at the 70^{th} percentile in weight, an eight-year old female who is at the 50^{th} percentile in height but at the 80^{th} percentile in weight, an eight-year old female who is at the 50^{th} percentile in height but at the 90^{th} percentile in weight, an eight-year old female who is at the 50^{th} percentile in height but at the 99^{th} percentile in weight, an adolescent female who is at the 50^{th} percentile in height but at the 60^{th} percentile in weight, an adolescent female who is at the 50^{th} percentile in height but at the 70^{th} percentile in weight, an adolescent female who is at the 50^{th} percentile in height but at the 80^{th} percentile in weight, an adolescent female who is at the 50^{th} percentile in height but at the 90^{th} percentile in weight, an adolescent female who is at the 50^{th} percentile in height but at the 99^{th} percentile in weight, an adult female who is at the 50^{th} percentile in height but at the 60^{th} percentile in weight, an adult female who is at the 50^{th} percentile in height but at the 70^{th} percentile in weight, an adult female who is at the 50^{th} percentile in height but at the 80^{th} percentile in weight, an adult female who is at the 50^{th} percentile in height but at the 90^{th} percentile in weight, an adult female who is at the 50^{th} percentile in height but at the 99^{th} percentile in weight, a middle-aged female who is at the 50^{th} percentile in height but at the 60^{th} percentile in weight, a middle-aged female who is at the 50^{th} percentile in height but at the 70^{th} percentile in weight, a middle-aged female who is at the 50^{th} percentile in height but at the 80^{th} percentile in weight, a middle-aged female who is at the 50^{th} percentile in height but at the 90^{th} percentile in weight, a middle-aged female who is at the 50^{th} percentile in height but at the 99^{th} percentile in weight, an elderly female who is at the 50^{th} percentile in height but at the 60^{th} percentile in weight, an elderly female who is at the 50^{th} percentile in height but at the 70^{th} percentile in weight, an elderly female who is at the 50^{th} percentile in height but at the 80^{th} percentile in weight, a elderly female who is at the 50^{th} percentile in height but at the 90^{th} percentile in weight, and an elderly female who is at the 50^{th} percentile in height but at the 99^{th} percentile in weight.

19. A kit comprising:
an apparatus comprising a belt comprising an interior surface; an upper long edge of said interior surface; a lower long edge of said interior surface; a first short edge of said interior surface; and, a second short edge of said interior surface; said belt further comprising an exterior surface; a lateral axis; a vertical axis; and a longitudinal axis; said belt further comprising a pocket defining an inner space, extending along a portion of the interior surface along the longitudinal axis of said belt, said pocket comprising four edges consisting of a first edge; a second edge; an upper edge; and, a lower edge wherein at least one of said four edges is affixed to said belt; said belt further comprising at least one fastener; and
at least one cold pack.

20. The kit of claim 19, further comprising a container of lotion wherein said lotion comprises at least one additive selected from the group consisting of additives configured to decrease the surface temperature of human epidermis cells, additives configured to increase metabolic activity in humans, additives configured to decreasing the sensitivity of human epidermis to temperature changes, additives configured to suppress appetite, and combinations thereof.

21. The kit of claim 20 further comprising a thermometer.

22. A method of cooling subcutaneous lipid-rich cells in a first region of epidermis of a subject, said method comprising the steps of:
disposing at least one cold pack into a pocket comprising an area of thin plastic wherein a first edge of said pocket is affixed to an interior surface of a belt and a second edge of said pocket is affixed to said interior surface of said belt;
contacting a first face of said at least one cold pack to the interior face of said area;
contacting said first region with said area so that heat is transferred from said region to said area;
positioning said interior surface of said belt along a second region of epidermis bounded by said first edge of said first region and by a second edge of said first region which is substantially parallel to said first edge of said first region such that said belt circumscribes said first region and said second region and such that said area contacts said first region; and,
fastening a first fastener which is affixed to a first end of the interior surface of said belt to a second fastener which is affixed to a second end of the exterior surface of said belt.

23. The method of claim 22 further comprising the step of applying to said first region a lotion comprising at least one additive selected from the group consisting of additives configured to decrease the surface temperature of human epidermis cells, additives configured to increase metabolic activity in humans, additives configured to decreasing the sensitivity of human epidermis to temperature changes, additives configured to suppress appetite, and combinations thereof.

24. The method of claim 23 further comprising the steps of:
waiting at least three minutes but no more than 45 minutes before removing said at least one cold pack from said pocket;
removing said at least one cold packet from said pocket;
rotating the first face of said at least one cold pack at least 25 degrees but not more than 330 degrees such that a second face of said at least one cold pack is aligned along the same plane as the interior face of said pocket; and,
inserting said at least one cold pack into said pocket such that said second face of said at least one cold pack contacts said inner face of said pocket and the portion of the inner of said pocket contacting said at least one cold pack also contacts said first region; and,
waiting no more than 45 minutes before moving said at least one cold pack to a position so that said at least one cold pack does not contact said first region.

25. The method of claim 24 wherein the step of disposing at least one cold pack into a pocket consists of disposing less than four cold packs into said pocket such that no more than three cold packs are simultaneously disposed within said pocket.

26. The method of claim 25, wherein said first region of epidermis resides on at least one body part of a human body selected from the group consisting of waists, torsos, trunks, hips, buttocks, chests, arms, forearms, thighs, knees, face, chins, legs, and calves.

27. The method of claim 26, further comprising the steps of:
inserting a thermometer between said pocket of said belt and said first region;
contacting said first region with said thermometer; and,
measuring a surface temperature of said first region of epidermis.

28. The method of claim 26, further comprising the steps of
inserting a thermometer between said pocket of said belt and said first region;
contacting said first region with said thermometer;
measuring a surface temperature of said first region of epidermis of greater than 40 degrees Fahrenheit but less than 60 degrees Fahrenheit;
rotating the first face of said cold pack at least 25 degrees but not more than 330 degrees such that said second face of said cold pack is aligned along the same plane as the interior face of said pocket; and,
inserting said cold pack into said pocket such that said second face of said cold pack contacts said inner face of said pocket.

29. The method of claim 27, further comprising the steps of:
removing said at least one cold pack from said pocket; and,
disposing a cold pack into said pocket.

30. The method of claim 26, further comprising the steps of
waiting at least five minutes before transporting said at least one cold pack;
transporting said at least one cold pack at least 0.5 inches to a second area disposed within said pocket;
contacting said first region with said thermometer;
measuring a surface temperature of said first region of epidermis of less than 40 degrees Fahrenheit;
removing said at least one cold pack from said pocket;
rotating the first face of said at least one cold pack at least 25 degrees but not more than 330 degrees such that said second face of said at least one cold pack is aligned along the same plane as the interior face of said pocket; and,
inserting said at least one cold pack into said pocket such that said second face of said at least one cold pack contacts said inner face of said pocket.

31. A method of cooling subcutaneous lipid-rich cells in a first region of epidermis of a subject, said method comprising the steps of:
applying a lotion to said first region;
wrapping a plastic wrap around a body part of said subject such that said plastic wrap covers said first region;
disposing at least one cold pack into a pocket comprising an area of thin plastic wherein a first edge of said pocket is affixed to an interior surface of a belt and a second edge of said pocket is affixed to said interior surface of said belt;
contacting a first face of said at least one cold pack to the interior face of said area;
contacting said first region with said area so that heat is transferred from said region to said area;
positioning said interior surface of said belt along a second region of epidermis bounded by said first edge of said first region and by a second edge of said first region which is substantially parallel to said first edge of said first region such that said belt circumscribes said first region and said second region and such that said area contacts said first region; and,
fastening a first fastener which is affixed to a first end of the interior surface of said belt to a second fastener which is affixed to a second end of the exterior surface of said belt.
